(19) United States
(12) Patent Application Publication
(10) Pub. No.: US 2008/0063702 A1
(43) Pub. Date: Mar. 13, 2008

(54) Liposomal Formulations Comprising an Amphipathic Weak Base Like Tempamine for Treatment of Neurodegenerative Conditions

(76) Inventors: Yechezkel Barenholz, Jerusalem (IL); Haim Ovadia, Jerusalem (IL); Pablo Kizelsztein, Yishuv Lapid (IL)

Correspondence Address: THE NATH LAW GROUP, 112 South West Street, Alexandria, VA, 22314, US

(21) Appl. No.: 11/662,173

(22) PCT Filed: Sep. 11, 2005
(86) PCT No.: PCT/IL05/00961

§ 371 Date: May 1, 2007

Related U.S. Application Data
(60) Provisional application No. 60/608,139, filed on Sep. 9, 2004.

(51) Int. Cl. A61K 9/127; A61K 31/45; A61P 25/00; A61P 25/16

(52) U.S. Cl. 424/450; 514/315

(57) ABSTRACT

The present invention provides the use of an amphipathic weak base having defined characteristics for the preparation of a pharmaceutical formulation for the treatment or prevention of neurodegenerative conditions. Preferably, the amphipathic weak base is encapsulated in a liposome. The invention also provides pharmaceutical formulations and methods of use thereof for the treatment or prevention of neurodegenerative conditions. A specific and preferred amphipathic weak base is tempamine (TMN). Further, preferably, tempamine is loaded in sterically stabilized liposomes (SSL-TMN).

### FIELD OF THE INVENTION

This invention generally concerns methods of treatment of neurodegenerative conditions, in particular by using drugs encapsulated by liposomes.

### PRIOR ART

The following is the prior art which is considered to be pertinent for describing the state of the art in the field of the invention.
WO03/053442;
Nichols, J. W., et al., Biochim. Biophys. Acta 455:269-271 (1976);
Cramer, J., et al., Biochemical and Biophysical Research Communications 75(2):295-301 (1977).

### BACKGROUND OF THE INVENTION

Neurodegenerative conditions, hereditary as well as sporadic conditions, are characterized by progressive nervous system dysfunction. These conditions are often associated with atrophy of the affected central or peripheral nervous system structures.

There is significant evidence that the pathogenesis of neurodegenerative diseases, including Parkinson's disease (PD) [Ebadi, M., et al. Prog. Neurobiol. 48(1):1-19 (1996)], Multiple Sclerosis (MS) [Lu F, et al. 177(2):95-103 (2000)], Alzheimer's disease (AD) [Markesbery, W. R. and Carney, J. M. Brain Pathol. 9:133-146 (1999)], Friedreich's ataxia [Sarsero J. P et al. J Gene Med. 5(1):72-81 (2003)], amyotrophic lateral sclerosis (ALS) [Ferrante, R. J., et al. J. Neurochem. 69(5):2064-2074 (1997)] and Huntington's disease (HD) [Borlongan, C. V., et. al. J. Fla. Med. Assoc. 83(5):335-341 (1996)] may be caused by the generation of reactive oxygen species (ROS). These are molecules which are not radicals in nature but are capable of radical formation in the extra- and intracellular environments such as hydrochlorous acid (HOCl), singlet oxygen (‘O₂) and hydrogen peroxide (H₂O₂). ROS are involved in many biological processes, including regulating biochemical processes, assisting in the action of specific enzymes, and removing and destroying bacteria and damaged cells. While free radicals are essential for the body for achieving a balance between oxidative and reductive compounds (redox state) inside the cell, if the balance is impaired in favor of oxidative compounds, oxidative stress (OS) occurs.

Accumulating data indicate that oxidative stress (OS) plays a major role in the pathogenesis of neurodegenerative diseases, such as MS, through the generation of ROS primarily by macrophages. As a result, demyelination and axonal damage are caused in both MS and experimental autoimmune encephalomyelitis (EAE, the acceptable animal model for MS).

There are many attempts to develop antioxidants that can cross the blood-brain barrier and decrease oxidative damage, leading to neurodegenerative conditions. Natural antioxidants such as vitamin E (tocopherol), carotenoids and flavonoids do not readily enter the brain in the adult, and the lazaroid antioxidant tirilazad (U-74006F) appears to localize in the blood-brain barrier. Thus, the use of modified spin traps and low molecular mass scavengers of O2*⁻ has been suggested [Halliwell B. Drugs Aging. 18(9):685-716 (2001)].

In addition to overcoming the blood-brain barrier, the fast clearance of antioxidants when administered in free form and their chemical degradation in plasma limit their effectiveness in vivo. Thus, a variety of approaches to extend the blood circulation time of these and other therapeutic agents have been developed. One such approach included the entrapment of the agent in a liposome.

There are a variety of drug-loading methods available for preparing liposomes with entrapped drug, including passive entrapment and active remote loading. The passive entrapment method is most suited for entrapping of lipophilic drugs which reside in the liposome's membrane and for entrapping drugs having high water solubility and/or high molecular weight. However, this method of loading is limited by the solubility of the drug in the hydration medium. In the case of ionizable amphipathic drugs, even greater drug-loading efficiency can be achieved by loading the drug into liposomes against a transmembrane ion gradient [Nichols, J. W., et al., Biochim. Biophys. Acta 455:269-271 (1976); Cramer, J., et al., Biochemical and Biophysical Research Communications 75(2):295-301 (1977)]. This loading method, generally referred to as remote loading, typically involves a drug which is amphipathic and has an ionizable amine group which is loaded by adding it to a suspension of liposomes having a higher inside/lower outside H⁺ or ionizable cation gradient (such as ammonium ions, for amphipathic weak bases) or having a lower inside/higher outside H⁺ or ionizable anion gradient (for amphipathic weak acids).

WO03/053442 describes a therapeutic formulation comprising tempamine (TMN) for the treatment of conditions caused by oxidative stress or cellular oxidative damage. The TMN is encapsulated in liposomes that provide an extended blood circulation lifetime for the drug. TMN release from liposomes, bio-distribution and pharmacokinetics of the liposome entrapped TMN are described.

### SUMMARY OF THE INVENTION

The present invention is based on several novel finding. Firstly, it was found that tempamine (an amphipathic weak base antioxidant at times referred to by the abbreviation, TMN) exhibits a protective effect on PC12 neurons against 1-Methyl, 4-phenyl, Pyridinium ion (MPP⁺) induced oxidative damage, and that the protective effect is in a dose dependent manner.

Further, it was found that two different liposomal formulations encapsulating, as the active ingredient, TMN, were significantly effective in reducing clinical signs of multiple sclerosis (MS) and Parkinson's disease (including incidence, duration and morbidity of the disease), in acceptable animal models. In the experiments conducted, sterically stabilized liposomes (SSL) encapsulating TMN (SSL-TMN) were used as TMN delivery system.

Yet further, it was found that the SSL-TMN formulations, having a diameter of about 80 nm, were more effective in penetrating the blood brain barrier (BBB) in experimental autoimmune encephalomyelitis (EAE, the acceptable animal model for MS) as compared to their penetration through the BBB of healthy animal.

Thus, it has been suggested that SSL-TMN may be of beneficial effect against neurodegenerative disorders, particularly those requiring penetration of a medication, through the blood brain barrier.

Thus, according to a first of its aspects, the present invention provides the use of an amphipathic weak base for the preparation of a pharmaceutical composition for the treatment or prevention of a neurodegenerative condition, the amphipathic weak base having one or more of the following characteristics: (i) it has pKa below 11.0; (ii) in an n-octanol/buffer (aqueous phase) system having a pH of 7.0, it has a partition coefficient in the range between about 0.001 and about 5.0, preferably in the range between about 0.005 and about 0.5; (iii) it exhibits an antioxidative activity; (iv) it exhibits a pro-apoptotic activity.

In accordance with another aspect of the invention, there is provided a pharmaceutical formulation for the treatment or prevention of a neurodegenerative condition comprising as an active ingredient an amphipathic weak base having one or more of the following characteristics: (i) it has pKa below 11.0; (ii) in an n-octanol/buffer (aqueous phase) system having a pH of 7.0, it has a partition coefficient in the range between about 0.001 and about 5.0, preferably in the range between about 0.005 and about 0.5; (iii) it exhibits an antioxidative activity; (iv) it exhibits a pro-apoptotic activity.

In yet another aspect of the invention there is provided a method of treating a subject having, or in disposition of developing a neurodegenerative condition, the method comprising administering to said subject an amount of pharmaceutical formulation comprising as active ingredient an amount of an amphipathic weak base having one or more of the following characteristics: (i) it has pKa below 11.0; (ii) in an n-octanol/buffer (aqueous phase) system having a pH of 7.0, it has a partition coefficient in the range between about 0.001 and about 5.0, preferably in the range between about 0.005 and about 0.5; (iii) it exhibits an antioxidative activity; (iv) it exhibits a pro-apoptotic activity.

Preferably, the amphipathic weak base is characterized by at least the above pKa and partition coefficient values.

The pharmaceutical composition should comprise a suitable physiologically and pharmaceutically acceptable carrier. Typically the carrier is such which allows the penetration of the active ingredient thought the blood brain barrier (BBB). Such penetration is important especially in neurodegenerative disease wherein the BBB remains un-damaged.

The carrier may be a molecule which is known to promote or facilitate entry through the BBB such as transferin receptor-binding agents, antibodies, or any drug that by itself transfers through the BBB. In such a case the molecule should be conjugated to the amphipathic weak acid of the invention by a bond which is cleavable in the BBB.

Another alternative is to incorporate the active ingredient in a suitable vehicle, such as lipid vesicles, nano-particles (coated or uncoated) or nano-capsules, effective to penetrate the BBB.

By a preferred embodiment the active ingredient is encapsulated in a lipid carrier, preferably a liposome as will be explained in more detail below.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a bar graph showing TMN protection in PC12 neurons against damage induced by MPP⁺. Cell death was evaluated by measuring the leakage of lactic dehydrogenase (LDH) into the medium.

FIG. 2 is a graph showing the effect of sterically stabilized liposomes loaded with TMN (SSL-TMN) on clinical signs (clinical score) of multiple sclerosis compared to that of commercially available drugs (Copaxone, Betaferon), when using an EAE model of the disease. Saline was used as control treatment.

FIG. 3 is a bar graph showing the pharmacokinetics in brain of healthy and EAE induced mice injected (i.v.) with [³H] Cholesteryl hexadecyl ether labelled SSL-TMN formulation.

FIG. 4A-4B are bar graphs showing the change in distribution of the SSL-TMN liposomes in healthy (FIG. 4A) and EAE induced mice (FIG. 4B) in the different tissues and in the plasma (plasma levels in FIG. 4A are divided in two).

FIG. 5 is a graph showing the effect of SSL-TMN on clinical signs (Mean clinical score) of multiple sclerosis compared to control treatment (Saline) when using another EAE model of the disease.

FIG. 6 is a graph showing the effect of treatment with SSL-TMN on 6-OHDA Parkinson induced animal model.

FIG. 7 is a bar graph showing the behavioral change of animals induced with 6-OHDA Parkinson after treatment with SSL-TMN (either i.v. or s.c. injection) or with control (saline).

### DETAILED DESCRIPTION OF THE INVENTION

The present invention concerns the use of an amphipathic weak base encapsulated in a pharmaceutically acceptable drug delivery vehicle, to form pharmaceutical formulations for treating neurodegenerative conditions.

The term “amphipathic weak base” is used herein to denote a molecule characterized by the following parameters:
(i) it has pKa below 11.0; preferably between about 11.0 and 7.5.
(ii) in an n-octanol/buffer (aqueous phase) system having a pH of 7.0, it has a partition coefficient in the range between about 0.001 and about 5.0, preferably in the range between about 0.005 and about 0.5.

These above characteristics are described in length in WO03/053442 (Table 2), incorporated herein in its entirety by reference.

The amphipathic weak base is further characterized by its biological activity, as an antioxidative agent and/or pro-apoptotic agent.

The term “antioxidant activity” or “antioxidative agent” refers to the fact that the amphipathic weak base is capable of interacting with free radicals, ROS and this are capable of preventing damage caused by free radicals

The term “pro-apoptotic activity” or “pro-apoptotic agent” refers to the fact that the amphipathic weak base is capable of inducing cell death via the induction of apoptosis [as described in WO03/053442].

According to one embodiment, the amphipathic weak base is a nitroxide compound. The term “nitroxide” is used herein to denote stable cyclic nitroxide free radicals, their precursors and their derivatives having a protonable amine, i.e. an amine capable of accepting at least one hydrogen proton. Non-limiting examples of cyclic nitroxides include carboxy nitroxides such as 5-carboxy-1,1,3,3-tetramethylisoindolin-2-yloxyl (CTMIO), 4-carboxy-2,2,6,6-tetramethylpiperidin-1-yloxyl (CTEMPO), and 3-carboxy-2,2,5,5-tetramethylpyrrolidin-1-yloxyl (CPROXYL), 2,2,6,6-tetramethyl-piperidine-1-oxyl (TEMPO), 4-hydroxy-2,2,6,6-tetramethylpiperidine-N-oxyl (TEMPOL), and -amino-2,2,2,6,6-tetramethyl-piperidine-N-oxyl (tempamine, TMN) A preferred group of cyclic nitroxides are piperidine nitroxides. A preferred amphipathic weak base in accordance with the invention which is a piperidine nitroxide is TMN.

In general, piperidine nitroxides, such as TEMPOL, TEMPO, and TMN are cell permeable, nontoxic and nonimmunogenic stable cyclic radicals [Afzal V. et al. Invest Radiol 19:549-552 (1984)]. Nitroxides exert their antioxidant activity through several mechanisms: SOD-mimic, oxidation of reduced metal ions, reduction of hypervalent metals and interruption of radical chain reactions [Samuni A. et al. Free Radic. Res. Commun. 12-13 Pt 1 187-197 (1991)]. Recently, piperidine nitroxides (Tempol and Tempo) were shown to possess anti-neoplastic activity and to enhance chemotherapy-induced apoptosis [Gariboldi et al. Free Radic. Biol. Med. 24:913-923 (1998); Shacter J A et al. Blood 96:307-313 (2000)].

The term “neurodegenerative conditions” is used herein interchangeably with the terms “neurodegenerative disease” and “neurodegenerative disorder” to denote any abnormal deterioration of the nervous system resulting in the dysfunction of the system. Further, it is used to denote a group of conditions in which there is gradual, generally relentlessly progressive wasting away of structural elements of the nervous system exhibited by any parameter related decrease in neuronal function, e.g. a reduction in mobility, a reduction in vocalization, decrease in cognitive function (notably learning and memory) abnormal limb-clasping reflex, retinal atrophy inability to succeed in a hang test, an increased level of MMP-2, an increased level of neurofibrillary tangles, increased tau phosphorylation, tau filament formation, abnormal neuronal morphology, lysosomal abnormalities, neuronal degeneration, gliosis and demyelination.

Without being limited thereto, neurodegenerative conditions may be classified according to the following groups:

Demyelinating and neuroautoimmune diseases, including, without being limited thereto acute, chronic progressive, and relapsing remitting multiple sclerosis (MS), Devic's disease, optic neuritis, acute disseminated encephalomyelitis, Guillain-Barre syndrome, chronic inflammatory demyelinating polyradiculoneuropathy, vasculitis, neural effect of systemic lupus erythematosus, neurosarcoidosis.

Infectious diseases, including, without being limited thereto cerebral malaria, post viral infectious encephalitis and Bell palsy.

Neurodegenerative disorders, including, without being limited thereto Alzheimer's disease, Parkinson's disease, senile dementias, prion diseases, spongiform encephalopathy, Creutzfeldt-Jakob disease, AIDS dementia, tauopathies and amyotrophic lateral sclerosis.

Brain Trauma, including, without being limited thereto, stroke, closed head injury, radiation injury and spinal cord trauma.

A preferred embodiment of the invention concerns the use of the amphipathic weak base as characterized above (preferably such as encapsulated in a liposome) for the preparation of a pharmaceutical formulation for treatment of multiple sclerosis (MS).

Another preferred embodiment of the invention concerns the use of the amphipathic weak base as characterized above (preferably such as encapsulated in a liposome) for the preparation of a pharmaceutical formulation for treatment of Parkinson's disease.

The terms “treat” or “treatment” are used herein to denote the administering of a an amount of the amphipathic weak base encapsulated in a pharmaceutically acceptable vehicle effective to prevent, inhibit or slow down abnormal deterioration of the nervous system, to ameliorate symptoms associated with a neurodegenerative condition, to prevent the manifestation of such symptoms before they occur, to slow down the irreversible damage caused by the chronic stage of the neurodegenerative condition, to lessen the severity or cure a neurodegenerative condition, to improve survival rate or more rapid recovery form such a condition. It should be noted that in the context of the present invention the term “treatment” also comprises prophylactic treatment i.e. for preventing deterioration of the nervous system and thereby development of a neurodegenerative conditions in subjects with high disposition of developing a neurodegenerative condition (as determined by considerations known to those versed in medicine) or for preventing the re-occurrence of an acute stage of a neurodegenerative condition in a chronically ill subjects. To this end, the vehicle loaded with the amphipathic weak base may be administered to subjects who do not exhibit a neurodegenerative condition but have a high-risk of developing such a condition, e.g. as a result of exposure to an agent which may cause abnormal generation of reactive oxidative species or subjects with family history of the disease (i.e. genetic disposition). In this case, the vehicle loaded with the amphipathic weak base will typically be administered over an extended period of time in a single daily dose (e.g. to produce a cumulative effective amount), in several doses a day, as a single dose for several days, etc. so as to prevent the damage to the nervous system.

The term “effective amount” is used herein to denote the amount of the amphipathic weak base when loaded in the vehicle in a given therapeutic regimen which is sufficient to inhibit or reduce the degradation of nerve cells and thereby the deterioration of the nervous system. The amount is determined by such considerations as may be known in the art and depends on the type and severity of the neurodegenerative condition to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the mode of administration, type of vehicle carrying the amphipathic weak base, the reactivity of the amphipathic weak base, its distribution profile within the body, a variety of pharmacological parameters such as half life in the body after being released from the vehicle, on undesired side effects, if any, on factors such as age and gender of the treated subject, etc.

It is noted that humans are treated generally longer than experimental animals as exemplified herein, which treatment has a length proportional to the length of the disease process and active agent effectiveness. The doses may be a single dose or multiple doses given over a period of several days.

While the following disclosure provides experimental data with animal model, there are a variety of acceptable approaches for converting doses from animal models to humans. For example, calculation of approximate body surface area (BSA) approach makes use of a simple allometric relationship based on body weight (W) such that BSA is equal to body weight (W) to the 0.67 power [Freireich E. J. et. al. Cancer Chemother. Reports 1966, 50(4) 219-244; and as analyzed in Dosage Regimen Design for Pharmaceutical Studies Conducted in Animals, by Mordenti, J, in J. Pharm. Sci., 75:852-57, 1986]. Further, allometry and tables of BSA data have been established [Extrapolation of Toxicological and Pharmacological Data from Animals to Humans, by Chappell W & Mordenti J, Advances in Drug Research, Vol. 20, 1-116, 1991 (published by Academic Press Ltd)]

Another approach for converting doses is a pharmacokinetic-based approach using the area under the concentration time curve (AUC) or Physiologically Based PharmacoKinetic (PBPK) methods are described [Voisin E. M. et al. Regul Toxicol Pharmacol. 12(2):107-116. (1990)]

The term “pharmaceutically/physiologically acceptable carrier” is used herein to denote any acceptable vehicle suitable for delivery of an active agent. Preferably it is a vehicle suitable to the delivery through the BBB. The vehicle may be a lipid based vesicle (e.g. liposomes) or a polymer based nanoparticle (e.g. where the polymer forms a matrix in which the amphipathic weak base may be embedded or a shell structure, where the amphipathic weak base is encapsulated within the core). Preferably, the vehicle is a liposome. Further, preferably, the carrier should be suitable for parenteral delivery of amphipathic weak bases, specifically, for administration by injection. Other modes of administration may include, without being limited thereto, oral, intranasal (e.g. using a polycationic lipid-based liposomes such as CCS described below), intra-ocular and topical administration as well as by infusion techniques)

The term “liposome” is used herein to denote lipid based bilayer vesicles. Liposomes are widely used as biocompatible carriers of drugs, peptides, proteins, plasmic DNA, antisense oligonucleotides or ribozymes, for pharmaceutical, cosmetic, and biochemical purposes. The enormous versatility in particle size and in the physical parameters of the lipids affords an attractive potential for constructing tailor-made vehicles for a wide range of applications. Different properties (size, colloidal behavior, phase transitions, electrical charge and polymorphism) of diverse lipid formulations (liposomes, lipoplexes, cubic phases, emulsions, micelles and solid lipid nanoparticles) for distinct applications (e.g. parenteral, transdermal, pulmonary, intranasal and oral administration) are available and known to those versed in the art. These properties influence relevant properties of the liposomes, such as liposome stability during storage and in serum, the biodistribution and passive or active (specific) targeting of cargo, and how to trigger drug release and membrane disintegration and/or fusion.

The liposomes are those composed primarily of liposome-forming lipids which are amphiphilic molecules essentially characterized by a packing parameter 0.74-1.0, or by a lipid mixture having an additive packing parameter (the sum of the packing parameters of each component of the liposome times the mole fraction of each component) in the range between 0.74 and 1. Liposome-forming lipids, exemplified herein by phospholipids, form into bilayer vesicles in water. The liposomes can also include other lipids incorporated into the lipid bilayers, with the hydrophobic moiety in contact with the interior, hydrophobic region of the bilayer membrane, and the head group moiety oriented toward the exterior, polar surface of the bilayer membrane.

The liposome-forming lipids are preferably those having a glycerol backbone wherein at least one, preferably two, of the hydroxyl groups at the head group is substituted with, preferably an acyl chain (to form an acyl or diacyl derivative), however, may also be substituted with an alkyl or allcenyl chain, a phosphate group or a combination or derivatives of same and may contain a chemically reactive group, (such as an amine, acid, ester, aldehyde or alcohol) at the headgroup, thereby providing a polar head group. Sphyngolipids, such as sphyngomyelins, are good alternative to glycerophopholipids.

Typically, the substituting chain(s), e.g. the acyl, alkyl or alkenyl chain is between 14 to about 24 carbon atoms in length, and has varying degrees of saturation being fully, partially or non-hydrogenated lipids. Further, the lipid may be of natural source, semi-synthetic or fully synthetic lipid, and neutral, negatively or positively charged. There are a variety of synthetic vesicle-forming lipids and naturally-occurring vesicle-forming lipids, including the phospholipids, such as phosphatidylcholine (PC), phosphatidylinositol (PI), phosphatidylglycerol (PG), dimyristoyl phosphatidylglycerol (DMPG); egg yolk phosphatidylcholine (EPC), 1-palmitoyl-2-oleoylphosphatidyl choline (POPC), distearoylphosphatidylcholine (DSPC), dimyristoyl phosphatidylcholine (DMPC); phosphatidic acid (PA), phosphatidylserine (PS) 1-palmitoyl-2-oleoylphosphatidyl choline (POPC) and the sphingophospholipids, such as sphingomyelin (SM) having 12-24 carbon atom acyl or alkyl chains. The above-described lipids and phospholipids whose hydrocarbon chain (acyl/alkyl/alkenyl chains) have varying degrees of saturation can be obtained commercially or prepared according to published methods. Other suitable lipids include in the liposomes are glyceroglycolipids and sphingoglycolipids and sterols (such as cholesterol or plant sterol).

Preferably, the phospholipid is egg phophatidylcholine (EPC), 1-palmitoyl-2-oleoylphosphatidyl choline (POPC), distearoylphosphatidylcholine (DSPC) or hydrogenated soy phosphatidylcholine (HSPC).

Cationic lipids (mono and polycationic) are also suitable for use in the liposomes of the invention, where the cationic lipid can be included as a minor component of the lipid composition or as a major or sole component. Such cationic lipids typically have a lipophilic moiety, such as a sterol, an acyl or diacyl chain, and where the lipid has an overall net positive charge. Preferably, the head group of the lipid carries the positive charge. Monocationic lipids may include, for example, 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy)propyl]-N,N,N-trimethylammonium chloride (DOTMA); 3β[N-(N′,N′-dimethylaminoethane)carbamoly]cholesterol (DC-Chol); and dimethyl-dioctadecylammonium (DDAB).

Examples of polycationic lipids include a similar lipophilic moiety as with the mono cationic lipids, to which polycationic moiety is attached. Exemplary polycationic moieties include spermine or spermidine (as exemplified by DOSPA and DOSPER), or a peptide, such as polylysine or other polyamine lipids. For example, the neutral lipid (DOPE) can be derivatized with polylysine to form a cationic lipid. polycationic lipids include, without being limited thereto, N-[2-[[2,5-bis[3-aminopropyl)amino]-1-oxopentyl]amino]ethyl]-N,N-dimethyl-2,3-bis[(1-oxo-9-octadecenyl)oxy]-1-propanaminium (DOSPA), and ceramide carbamoyl spermine (CCS).

The lipids mixture forming the liposome can be selected to achieve a specified degree of fluidity or rigidity, to control the stability of the liposome in serum and to control the rate of release of the entrapped agent in the liposome.

Further, the liposomes may also include a lipid derivatized with a hydrophilic polymer to form new entities known by the term lipopolymers. Lipopolymers preferably comprise lipids, modified at their head group with a polymer having a molecular weight equal or above 750 Da. The head group may be polar or apolar, however, is preferably a polar head group to which a large (>750 Da) highly hydrated (at least 60 molecules of water per head group) flexible polymer is attached. The attachment of the hydrophilic polymer head group to the lipid region may be a covalent or non-covalent attachment, however, is preferably via the formation of a covalent bond (optionally via a linker). The outermost surface coating of hydrophilic polymer chains is effective to provide a liposome with a long blood circulation lifetime in vivo. The lipopolymer may be introduced into the liposome by two different ways: (a) either by adding the lipopolymer to a lipid mixture forming the liposome. The lipopolymer will be incorporated and exposed at the inner and outer leaflets of the liposome bilayer [Uster P. S. et al. FEBBS Letters 386:243 (1996)]; (b) or by firstly prepare the liposome and then incorporate the lipopolymers to the external leaflet of the pre-formed liposome either by incubation at temperature above the Tm of the lipopolymer and liposome-forming lipids, or by short term exposure to microwave irradiation.

Preparation of vesicles composed of liposome-forming lipids and derivatization of such lipids with hydrophilic polymers (thereby forming lipopolymers) has been described, for example by Tirosh et al. [Tirosh et al., Biopys. J., 74(3):1371-1379, (1998)] and in U.S. Pat. Nos. 5,013,556; 5,395,619; 5,817,856; 6,043,094, 6,165,501, incorporated herein by reference and in WO 98/07409. The lipopolymers may be non-ionic lipopolymers (also referred to at times as neutral lipopolymers or uncharged lipopolymers) or lipopolymers having a net negative or a net positive charge.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers.

While the lipids derivatized into lipopolymers may be neutral, negatively charged, as well as positively charged, i.e. there is no restriction to a specific (or no) charge, the most commonly used and commercially available lipids derivatized into lipopolymers are those based on phosphatidyl ethanolamine (PE), usually, distearylphosphatidylethanolamine (DSPE).

A specific family of lipopolymers employed by the invention include monomethylated PEG attached to DSPE (with different lengths of PEG chains, the methylated PEG referred to herein by the abbreviated PEG) in which the PEG polymer is linked to the lipid via a carbamate linkage resulting in a negatively charged lipopolymer. Other lipopolymers are the neutral methyl polyethyleneglycol distearoylglycerol (mPEG-DSG) and the neutral methyl polyethyleneglycol oxycarbonyl-3-amino-1,2-propanediol distearoylester (mPEG-DS) [Garbuzenko O. et al., Langmuir. 21:2560-2568 (2005)]. The PEG moiety preferably has a molecular weight of the head group is from about 750 Da to about 20,000 Da. More preferably, the molecular weight is from about 750 Da to about 12,000 Da and most preferably between about 1,000 Da to about 5,000 Da. One specific PEG-DSPE employed herein is that wherein PEG has a molecular weight of 2000 Da, designated herein ²⁰⁰⁰PEG-DSPE or ^{2k}PEG-DSPE.

Preparation of liposomes including such derivatized lipids has also been described, where typically, between 1-20 mole percent of such a derivatized lipid is included in the liposome formulation.

As discussed above, the amphipathic weak base is preferably used in combination with a vehicle. According to a preferred embodiment, the vehicle is a lipid vesicle, and amphipathic weak base is encapsulated within the vesicle. more preferably, the vesicle is a liposome.

The term “encapsulating” is used herein to denote the loading of the amphipathic weak base into the aqueous phase of the lipid vesicle, e.g. liposome. Loading is preferably achieved the use of remote loading techniques where the antioxidant is loaded into pre-formed liposomes by loading against an ammonium ion concentration gradient, as has been described in U.S. Pat. No. 5,192,549. According to this method the amphipathic weak base is accumulated in the intraliposome aqueous compartment at concentration levels much greater than can be achieved by other loading methods.

As used herein, “administering” is used to denote the contacting or dispensing, delivering or applying the amphipathic weak base, preferably carried by a vehicle, to a subject by any suitable route for delivery thereof to the desired location in the subject, preferably by the parenteral route including subcutaneous, intramuscular and intravenous, intraarterial, intraperitoneally as well as by intranasal administration, intrathecal and infusion techniques.

According to one preferred embodiment, the formulations used in accordance with the invention are in a form suitable for injection. The requirements for effective pharmaceutical vehicles for injectable formulations are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

A preferred embodiment of the invention concerns liposomes comprising between 1 to 20 mole percent of a lipopolymer. A preferred hydrophilic moiety of the lipopolymer is PEG and a preferred dervatized lipopolymer is either ²⁰⁰⁰PEG-DSPE ²⁰⁰⁰PEG-DS or ²⁰⁰⁰PEG-DSG.

Variations in ratios between these liposome constituents dictate the pharmacological properties of the liposome, including stability of the liposomes, which is a major concern for various types of vesicular applications. Evidently, the stability of liposomes should meet the same standards as conventional pharmaceuticals. Chemical stability involves prevention of both the hydrolysis of ester bonds in the phospholipid bilayer and the oxidation of unsaturated sites in the lipid chain. Chemical instability can lead to physical instability or leakage of encapsulated drug from the bilayer and fusion and aggregation of vesicles. Chemical instability also results in short blood circulation time of the liposome, which affects the effective access to and interaction with the target.

Specific liposomes compositions according to the invention are those comprising a liposome forming lipid, such as hydrogenetaed soy phosphatidylcholine (HSPC) or egg phosphatidylcholine (EPC), in combination with cholesterol (Chol) and said lipopolymer. Specific embodiments include the following liposome compositions: EPC:Chol:²⁰⁰⁰PEG-DSPE and HSPC:Chol:²⁰⁰⁰PEG-DSPE both in a mole ratio of 54:41:5. Evidently, other liposome forming lipids may be utilized in the same or similar mole ratio, and provided that the final additive packing parameter of the different constituents of the liposome is in the range of between about 0.74 and 1.0.

According to a preferred embodiment of the invention pre-formed liposomes are used for remote loading of the amphipathic weak base, against an ion concentration gradient, into the liposomes. Liposomes having an H⁺ and/or ion gradient across the liposome bilayer for use in remote loading can be prepared by a variety of techniques. A typical procedure comprises dissolving a mixture of lipids at a ratio that forms stable liposomes in a suitable organic solvent and evaporated in a vessel to form a thin lipid film. The film is then hydrated with an aqueous medium containing the solute species that will form the intra-liposome aqueous phase and will also serve the basis for the ion transmembrane gradient (inner liposome high/outer medium low).

After liposome formation, the liposomes may be sized to achieve a size distribution of liposomes within a selected range, according to known methods. The liposomes are preferably uniformly sized to a selected size range between 70-100 nm, preferably about 80 nm.

After sizing, the external medium of the liposomes is treated to produce an ion gradient across the liposome membrane, which is typically a higher inside/lower outside ion concentration gradient. This may be done in a variety of ways, e.g., by (i) diluting the external medium, (ii) dialysis against the desired final medium, (iii) gel exclusion chromatography, e.g., using Sephadex G-50, equilibrated in the desired medium which is used for elution, or (iv) repeated high-speed centrifugation and resuspension of pelleted liposomes in the desired final medium. The selection of the external medium will depend on the mechanism of gradient formation, the external solute and pH desired, as will now be considered.

In the simplest approach for generating an ion and/or H⁺ gradient, the lipids are hydrated and sized in a medium having a selected internal-medium pH. The suspension of the liposomes is titrated until the external liposome mixture reaches the desired final pH, or treated as above to exchange the external phase buffer with one having the desired external pH. For example, the original hydration medium may have a pH of 5.5, in a selected buffer, e.g., glutamate, citrate, succinate, fumarate buffer, and the final external medium may have a pH of 8.5 in the same or different buffer. The common characteristic of these buffers is that they are formed from acids which are essentially liposome impermeable. The internal and external media are preferably selected to contain about the same osmolarity, e.g., by suitable adjustment of the concentration of buffer, salt, or low molecular weight non-electrolyte solute, such as dextrose or sucrose.

In another general approach, the gradient is produced by including in the liposomes, a ion selective ionophore. To illustrate, liposomes prepared to contain valinomycin in the liposome bilayer are prepared in a potassium buffer, sized, then the external medium exchanged with a sodium buffer, creating a potassium inside/sodium outside gradient. The K⁺ selective ionophore valinomycin enables movement of potassium ions in an inside-to-outside direction in turn generates a lower inside/higher outside pH gradient, presumably due to movement of protons into the liposomes in response to the net electronegative charge across the liposome membranes [Deamer, D. W., et al., Biochim. et Biophys. Acta 274:323 (1972)].

A similar approach is to hydrate the lipid and to size the formed multilamellar liposome in high concentration of magnesium sulfate. The magnesium sulfate gradient is created by dialysis against 20 mM HEPPES buffer, pH 7.4 in sucrose. Then, the A23187 ionophore is added, resulting in outwards transport of the magnesium ion in exchange for two protons for each magnesium ion, plus establishing a inner liposome high/outer liposome low proton gradient [Senske D B et al. (Biochim. Biophys. Acta 1414: 188-204 (1998)].

In another more preferred approach, the proton gradient used for drug loading is produced by creating an ammonium ion gradient across the liposome membrane, as described, for example, in U.S. Pat. Nos. 5,192,549 and 5,316,771, incorporated herein by reference. The liposomes are prepared in an aqueous buffer containing an ammonium salt, such as ammonium sulfate, ammonium phosphate, ammonium citrate, etc., typically 0.1 to 0.3 M ammonium salt, at a suitable pH, e.g., 5.5 to 7.5. The gradient can also be produced by including in the hydration medium sulfated polymers, such as dextran sulfate ammonium salt, heparin sulfate ammonium salt or sucralfate. After liposome formation and sizing, the external medium is exchanged for one lacking ammonium ions. In this approach, during the loading the amphipathic weak base is exchanged with the ammonium ion.

Yet, another approach is described in U.S. Pat. No. 5,939,096, incorporated herein by reference. In brief, the method employs a proton shuttle mechanism involving the salt of a weak acid, such as acetic acid, of which the protonated form trans-locates across the liposome membrane to generate a higher inside/lower outside pH gradient. An amphipathic weak acid compound is then added to the medium to the pre-formed liposomes. This amphipathic weak acid accumulates in liposomes in response to this gradient, and may be retained in the liposomes by cation (i.e. calcium ions)-promoted precipitation or low permeability across the liposome membrane, namely, the amphipathic weak acid is exchanges with the acetic acid.

The use of remote loading and in particular the latter ammonium ion gradient procedure enables high loading of the amphipathic weak base into the liposome. A preferred amphipathic weak base to lipid ratio is in the range of between about 0.01 to about 2 and preferably between about 0.001 to about 4, preferably between 0.01 to about 2. For high loading of the amphipathic weak base it is at times preferable that the concentration of the same in the liposome be such that it precipitates in the presence of a co-entrapped counter ion, such as sulfate.

According to another preferred embodiment, the loading of the amphipathic weak base should be performed at a temperature range of the gel to liquid crystalline phase transition.

The present invention preferably concerns the use of liposomal formulations comprising a cyclic nitroxide as the amphipathic weak base. A preferred amphipathic weak base is a cyclic nitroxide is TMN.

Thus, a preferred liposomal formulation according to the invention is TMN encapsulated in sterically stabilized liposomes (SSL). In order to penetrate at sufficient level the blood brain barrier, it is essential that the SSL have a diameter of about 80 nm or smaller.

The following examples further illustrate the invention described herein and are in no way intended to limit the scope of the invention.

### DETAILED DESCRIPTION OF EXEMPLARY EMBODIMENTS

### EXAMPLE 1

### TMN Effect on Neurons

Cell Culture

PC12 cells were grown in Dulbecco's modified Eagle's medium (DMEM), supplemented with 7% fetal calf serum, 7% horse serum, 100 μg/ml streptomycin, and 100 U/ml penicillin. The cultures were maintained in an incubator at 37° C. in a humidified atmosphere of 6% CO₂. The growth medium was changed twice weekly and the cultures were split at 1:6 ratio once a week [Abu-Raya et al. J. Neurosci. Methods 50:197-203 (1993)].

For differentiation, an identical number of PC12 cells (3.75×10⁵ cells) was plated on 6-wells plates coated with rat tail type I collagen (0.1 mg/ml) to promote cell adhesion [Abu-Raya et al Rasagiline, a monoamine oxidase-B inhibitor, protects NGF-differentiated PC12 cells against oxygen-glucose deprivation. J. Neurosci. Res. 58:456-463 (1999)]. The differentiation of the cultures was induced by treatment with NGF (50 ng/ml), added every 48 hr for a period of 7-8 days.

Measurement of Cell Death-Lactate Dehydrogenase (LDH) Leakage Assay

Cell death was evaluated by measuring the leakage of LDH into the growth medium as previously described [Abu-Raya et al. J. Neurosci. Res. 58:456-463, (1999)]. Samples of 50 μl of the growth medium were collected from each well and centrifuged at 3,500 rpm for 5 min at 25° C.; the supernatant was collected and LDH release was measured using a TRACE LD-L reagent. LDH activity was determined using an ELISA reader (TECAN, SPECTRAFluor PLUS, Grodig, Salzburg, Austria) at 340 nm by following the rate of conversion of oxidized nicotinamide adenine dinucleotide (NAD⁺) to the reduced form of (NADH). MPP⁺-induced LDH release was expressed as 100% of toxicity compared to control-untreated cultures. Each experiment was performed three times in duplicates (n=6).

MPP⁺ Toxicity Experiment

On the day of the experiment, the NGF containing medium was replaced with fresh one. The cultures were divided into the three groups: 1) control—untreated cells; 2) cultures exposed to MPP⁺ insult; 3) TMN treated cultures exposed to MPP⁺ insult.

MPP⁺ was dissolved in growth medium containing NGF and added to each well in a final concentration of 1500 μM. At the end of experiment, medium was taken for evaluation of LDH release. During the experiment, all cultures were maintained in an incubator at 37° C. in a humidified atmosphere of 6% CO₂. The experiment was accomplished when percentage of cell death was in the range 30-60%, measured by the release of LDH into the medium.

TMN dissolved in growth medium containing NGF, was added to the cultures 1 hr prior to the exposure to MPP⁺. For dose response assay, TMN was administered to each well in a final concentrations of 0.1, 1, 10, 100, 500 or 1000 μM. Samples of 50 μl medium were taken after 48 hr for assessment of LDH release.

Results

Tempamine Protective Effects in PC12 Neurons Exposed to Damage by MPP⁺

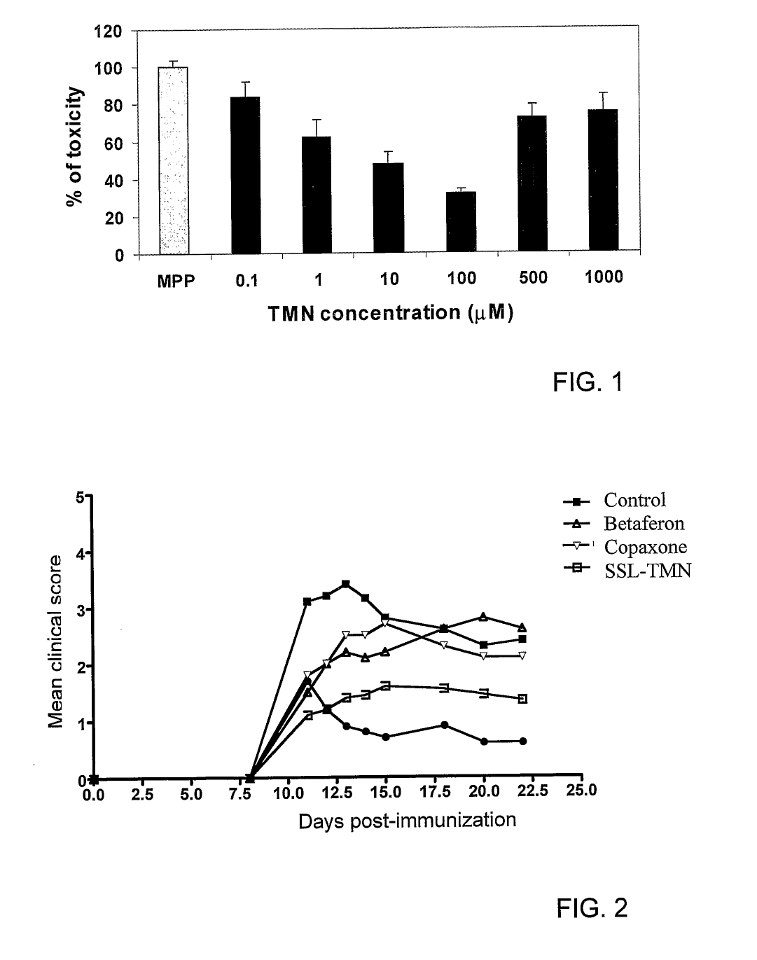

FIG. 1 demonstrates that TMN protects PC12 neurons from oxidative damage inflicted by 1500 μM MPP⁺ in a dose dependent manner in the range of (0.1-100 μM), with 100 μM being most effective. The bell shape at higher concentration 500 μM-1000 μM (irrelevant concentrations for therapeutic applications) may imply that at higher concentration TMN is toxic to the cells.

### EXAMPLE 2

### Liposomal Formulations Comprising TMN

Materials and Methods

2,2,6,6-tetramethylpiperidine-4-amino-1-oxyl (4-amino-tempo, termed TMN, TMN) free radical, 97%, was purchased from Aldrich (Milwaukee, Wis., USA).

Egg phosphatidylcholine (EPC I) and hydrogenated soybean phosphatidylcholine (HSPC) were obtained from Lipoid KG (Ludwigshafen, Germany).

N-carbamyl-poly-(ethylene glycol methyl ether)-1,2-distearoyl-s-n-glycero-3-phosphoethanolamine triethyl ammonium salt (²⁰⁰⁰PEG-DSPE) was obtained from Genzyme (Lista, Switzerland).

Cholesterol was obtained from Sigma (St. Louis, Mo., USA).

Sephadex G-50 was obtained from Pharmacia (Uppsala, Sweden).

tert-Ethanol was purchased from BDH, Poole, UK.

Fluoroscein phosphatidylethanolamine (F-PE) was obtained from Avanti Polar Lipids (Alabaster, Ala., USA).

Other chemicals, including buffers, were obtained from Sigma. Dialysis membrane (dialysis tubing-visking (size 6- 27/32”) was obtained from Medicell International (London, UK).

Purified water (WaterPro PS HPLC/Ultrafilter Hybrid model, Labconco, Kansas City, Mo., USA) which provides lowest possible levels of total organic carbon and inorganic ions was used in all water-based preparations (resistance of 18.2 megaohm).

Electron Paramagnetic Resonance (EPR) Measurements

EPR spectrometry was employed to detect TMN concentration using a JES-RE3X EPR spectrometer (JEOL Co., Japan) (Fuchs, J., et al., Free Radic. Biol. Med. 22:967-976, (1997)). Samples were drawn by a syringe into a gas-permeable Teflon capillary tube of 0.81 mm i.d. and 0.05 mm wall thickness (Zeus Industrial Products, Raritan, N.J., USA). The capillary tube was inserted into a 2.5-mm-i.d. quartz tube open at both ends, and placed in the EPR cavity. EPR spectra were recorded with center field set at 329 mT, 100 kHz modulation frequency, 0.1 mT modulation amplitude, and nonsaturating microwave power. Just before EPR measurements, loaded liposomes were diluted with 0.15 M NaCl for the suitable TMN concentration range (0.02-0.1 mM). The experiment was carried out under air, at room temperature. This is a functional assay which determines the activity of TMN.

Cyclic Voltammetry (CV) Measurements

All cyclic voltammograms were performed between—200 mV and 1.3 V. Measurements were carried out in phosphate-buffered saline, pH 7.4. A three-electrode system was used throughout the study. The working electrode was a glassy carbon disk (BAS MF-2012, Bioanalytical Systems, W. Lafayette, Ind., USA), 3.3 mm in diameter. The auxiliary electrode was a platinum wire, and the reference electrode was Ag/AgCl (BAS). The working electrode was polished before each measurement using a polishing kit (BAS PK-1) (Kohen, R., et al., Arch. Gerontol. Geriatr., 24:103-123, (1996)). Just before CV measurements the samples were diluted with buffer to the optimal TMN concentration range (0.05-0.2 mM). The experiments were carried out under air, at room temperature. The CV assay is a functional assay determining the ability of the analyte to accept or donate electrons.

Liposome Preparation

Liposome Formation

A stock solution of EPC, Cholesterol and ²⁰⁰⁰PEG-DSPE at a mole ratio of 54:41:5 was mixed in ethanol at 70° C. to reach a final lipid concentration of 62.5% (w/v), then incubated at 70° C. for 15 min until all the lipids were dissolved and a clear solution was obtained. The ethanol stock solution containing lipid was then added to a solution of 250 mM ammonium sulfate at 70° C. to reach a final lipid concentration of 6.25% (w/v) reaching a final ethanol concentration of 10% (w/v). The mixture was constantly stirred at 70° C. until a milky dispersion was obtained, at this stage lipids were hydrated to form un-sized heterogeneous multillamellar liposomes (MLVs).

Also the approach of lyophilization from tertiary buthanol (freezing temperature of 22° C.) followed by mechanical hydration (vortexing) and extrusion was used [G. Haran et al. Biochim Biophys. Acta 1151:201-215 (1993)]. All lipids were dissolved in tert-butanol and lyophilized overnight. The dry lipid powder was hydrated with ammonium sulfate solution (150 mM). Hydration was carried out above the Tₘ of the matrix lipid: for HSPC, 60° C. (Tm=52.2° C.) and for EPC room temperature, (Tm=−5° C.). Hydration was performed under continuous shaking, forming multilamellar vesicles (MLV). The volume of hydration medium was adjusted to obtain a 10% (w/v) lipid concentration. Large unilamellar vesicles (LUV 100 nm) were prepared by stepwise extrusion using a 100-nm-pore-size polycarbonate filter as the last step.

The liposome size distribution was determined by dynamic light scattering (DLS) using either a Coulter (Model N4 SD) submicron particle analyzer or ALV-NIBS/HPPS with ALV-5000/EPP multiple digital correlator (ALV-Laser Vertriebsgesellschaft GmbH, Langen, Germany [Barenholz Y and Amselem S. Liposome Technology 2nd Edition, Vol I, Liposome Preparation and Related Techniques 527-616 (1993)]. Size distributions of 1200±200 nm (polymodal) and 100±10 nm (unimodal) were obtained for MLV and LUV, respectively.

Small unilamellar vesicles (SUVs) were obtained by stepwise extrusion through double-stacked polycarbonate membranes of gradually decreasing pore size (0.4,0.1,0.08 and 0.05 um) using a high pressure extrusion device (Lipex Biomembranes, Vancouver,BC,Canada) TMN was remote loaded actively into the thus pre-formed SUV by the use of ammonium sulfate gradient as described below.

Formation of Ammonium Sulfate Gradient

For the formation of ammonium sulfate gradient the dialysis procedure of Amselem et al. [Amselem et al. J. Liposome Res., 2:93-123 (1992)] was utilized. In brief, the procedure used two or three consecutive dialysis exchanges (dialysis tubing-visking (size 9 36/32”) from Medicell International each against 100 volumes of 0.13M NaCl 0.01M Na citrate (pH=7.4).

Liposome Loading with Tempamine

Liposome loading with TMN was performed as described in WO03/053442. Briefly, a concentrated TMN alcoholic solution (0.8 ml of 25 mM TMN in 70% ethanol) was added to 10 ml of liposomal suspension. The final solution contained 5.6% ethanol and 2 mM TMN. Loading was performed above the Tₘ of the matrix lipid. Loading was terminated at the specified time by removal of non-encapsulated TMN using the dialysis at 4° C.

Loading efficiency was determined as described below.

Percent Encapsulation of Tempamine

The amount of entrapped TMN in liposomes prepared was determined as described in WO03/053442 using either EPR or CV. For EPR measurements first, the total TMN in the post-loading liposome preparation (TMNₘᵢₓ) was measured. Then, the amount of TMN in the post-loading liposome preparation in the presence of potassium ferricyanide, an EPR broadening agent that eliminates the signal of free (non-liposomal) TMN, was measured. The remaining signal was of TMN in liposomes (TMN_{liposome(quenched)}). The resulting spectrum was broad, as TMN concentration inside the liposomes was high, leading to quenching of its EPR signal due to spin interaction between the TMN molecules which are close to one another. Then the total TMN after releasing it from liposomes by nigericin (TMN_{nigericin}) was measured. This signal was identical to the total TMN used for loading (TMN_{nigericin}=TMNₜₒₜₐₗ) and is completely dequenched. TMN_{liposome(not quenched)} represents the signal of liposomal TMN when the ammonium sulfate gradient is collapsed and all the TMN is released.

The percent encapsulation and the quenching factor were calculated as follows:
TMN_{free}TMNₘᵢₓ−TMN_{liposomes(quenched)} (1)
TMN_{liposomes(not quenched)}=TMN_{nigericin}-TMN_{free} (2)
Percent encapsulation=100×TMN_{liposome(not quenched)}/TMN_{nigericin} (3)
Quenching factor=TMN_{liposome(not quenched)}/TMN_{liposome(quenched)} (4)

The data are summarized in Table 1.

The level of TMN total=TMN nigericin agreed well with the TMN determined after liposome solubilization by 1% Triton X-100. For TMN determination by CV. firstly free TMN (remaining after loading into liposomes) was determined. From these, level of free TMN, and percent TMN encapsulated were calculated. There was a good agreement between EPR and CV measurements as also described in WO03/053442.

TABLE 1
Lipid composition in liposomes

| | | | TMN/ |
|---|---|---|---|
| | | % | phospholipids |
| No. | Liposome composition^{(a)} | Encapsulation^{(b)} | ratio |
| 1. | EPC | 85 | 0.09 |
| 2. | HSPC | 85 | 0.09 |
| 3. | EPC:Chol:²⁰⁰⁰PEG-DSPE (54:41:5)^{(c)} | 96 | 0.12 |
| 4 | HSPC:Chol:²⁰⁰⁰PEG-DSPE (54:41:5)^{(c)} | 96 | 0.12 |

^{(a)}EPC—egg phosphatidylcholine; HSPC—hydrogenated soy phosphatidylcholine; Chol—cholesterol; ²⁰⁰⁰PEG-DSPE—N-carbamyl-poly-(ethylene glycol methyl ether)- 1,2-distearoyl-s-n-glycero-3-phosphoethanolamine triethyl ammonium salt
^{(b)}percent encapsulation determined by CV and confirmed by EPR (see above) when applying the remote loading procedure
^{(c)}in mole ratio

Nitroxide Quantification

TMN concentration in tissues, brain and plasma was quantified using electron paramagnetic resonance (EPR) in the presence of 1.32% Triton X-100 that solubilize the liposomes and enables detection of encapsulated and free TMN levels, as described in the above methods section.

Phospholipid Concentration

Phospholipids concentration in the liposome composition was determined using a modification of Bartlett's procedure [Barenholz Y. et al. in LIPOSOME TECHNOLOGY, G. Gregoriadis (Ed.) 2^{nd} Edn, Vol I, CRC Press, Boca Raton 527-616 (1993);, Shmeeda et al, Methods in Enzymol. 367:272-292 (2003)]

Dosage Form

Free TMN (a concentrated TMN alcoholic solution (500 mM TMN in 70% ETOH) was diluted in saline to obtain an 10 mM concentration or was added to liposomes (EPC:Chol:²⁰⁰⁰PEG-DSPE) to reach a final concentration of 10 nM TMN.

Liposomes Biodistribution:

Six to 7-week-SJL female mice, obtained through the Animal Breeding House of the Hebrew University (Jerusalem, Israel), were used throughout the biodistribution experiment. Animals were housed at Hadassah Medical Center at an SPF faculty with food and water ad libitum. The experimental procedures were in accordance with the standards required by the Institutional Animal Care and Use Committee of the Hebrew University and Hadassah Medical Organization.

SSL liposomes composed of EPC:Chol:²⁰⁰⁰PEG-DSPE (54:41:5) mole ratio, and a trace amount of [³H] cholesteryl ether (0.5 μCi/μmol phospholipid) were prepared as described by Kedar et al [Kedar et al, J Immunother Emphasis Tumor Immunol. 16(1):47-59 (1994)]. At 1, 6, 16, 24, 48 h and 72 h after the [³H] Cholesteryl hexadecyl ether SSL-TMN IV injection, the animals were anesthetized with ether inhalation, bled from the orbital sinus, and immediately sacrificed for removal of brain, heat, lungs, liver, spleen, stomach and kidney. Each time point consisted of 2 mice. Plasma was separated from blood cells by centrifugation.

Organs were homogenized in a Polytron homogenizer (Kinematica, Lutzem, Switzerland) in 2% Triton X-100 (1:2, organ:Triton X-100 solution), cooled and heated several times to release the TMN. The plasma samples were mixed 1:1 with 2% Triton X-100 to give the 1% Triton X-100 in the tested sample and also cooled and heated several times. Under such conditions it was determined that intact liposomes released all their TMN (for further TMN determinations).

Sample duplicates of 100 μl were burned in a Sample Oxidizer (Model 307, Packard Instrument Co., Meridien, Conn.) left overnight in a dark, cool place and measured by β-counting (KONTRON Liquid Scintillation Counter). Radiospecific activity of the liposomes DPM/μmole was calculated.

### EXAMPLE 2A

### Multiple Sclerosis (MS)

Animal Model

A. Induction of Acute EAE Using PLP (Proteolipid Protein)

Induction of EAE using proteolipid protein was performed as described in Pollak J of Neuroimmunology 137:94-99 (2003)]. In brief, 6-7 week old SJL female mice were immunized by subcutaneous injection in the right flank with an emulsion containing proteolipid protein (PLP) 139-151 peptide and complete Freund's adjuvant (CFA) containing 150 μg of peptide and 200 μg of Mycobacterium tuberculosis. On the day of the first PLP injection, Pertussis Toxin (PT) 150 ng was injected intraperitoneally (0.1 ml/mice).

The animals were kept in specific pathogen free (SPF) conditions and given food ad libitum.

For treatment, the animals (10 mice per group) were divided into groups and treated as summarized in Table 2 below.

TABLE 2
Schedule of treatment

| | | | | Regime of |
|---|---|---|---|---|
| Group | Treatment formulation | Dose | | administration |
| 1 | Control (Saline) | 45 | mg/kg | s.c. × 3/week |
| 2 | Betaferon | 0.007 | mg/kg ( | s.c. × 3/week |
| 3 | Copaxone | 12.5 | mg/kg | s.c. × 3/week |
| 4 | SSL-TMN | 8.5 | mg/kg | i.v. × 3/week |
| 5 | SSL-TMN | 8.5 | mg/kg | s.c. × 3/week |

Once clinical signs of MS appeared (i.e. on day 10 post inoculation with PLP), the mice received treatment either with a conventional MS medication such as Betaferon (Schering AG Germany) or Copaxone (Teva pharmaceuticals, Israel), or with the sterically stabilized TMN formulation (EPC:Chol:²⁰⁰⁰PEG-DSPE, 54:41:5, SSL-TMN in Table 2 below) described in Table 1 above.

The mice were observed daily from the 10th day post-EAE induction (PLP injection, i.e. the first day of treatment) and the EAE clinical signs were scored. The scores were performed according to Table 3 below:

TABLE 3
clinical signs scoring

| Score | Signs | Description |
|---|---|---|
| 0 | Normal behavior | No neurological signs |
| 1 | Distal limp tail | The distal part of the tail is limp |
| | | and droops |
| 1.5 | Complete limp tail | The whole tail is loose and droops |
| 2 | Complete limp | The whole tail is loose and droops. |
| | tail with | Animal has difficulties to return on |
| | righting reflex | his feet when it is laid on his back |
| 3 | Ataxia | Woobly walk- when the mouse walks |
| | | the hind legs are unsteady |
| 4 | Early paralysis | The mouse has difficulties standing |
| | | on its hind legs but still has |
| | | remnants of movement |
| 5 | Full paralysis | The mouse can't move its legs at |
| | | all, it looks thinner |
| | | and emaciated. Incontinence |
| 6 | Moribund/death | |

The number of mice in each animal group which developed the disease (sick) was summed and the percentage thereof was calculated.

In addition, the mean maximal score (MMS) by summing the maximal scores of each of the 10 mice in the group and calculating therefrom the mean maximal score of the group according to the following equation:
Σmaximal score of each mouse/number of mice in the group

Further, the mean duration of disease (MDD) expressed in days was calculated according to the following equation:
Σduration of disease of each mouse/number of mice in the group

Further, each group's mean score (GMS) (burden of disease) was determined by summing the scores of each of the 10 mice in the group and calculating the mean score per day, according to the following equation:
Σtotal score of each mouse per day/number of mice in the group.

Tables 4A, 4B and 4C (obtained from three separate assays) and FIG. 1 summarize the different scores calculated:

TABLE 4A
clinical signs scores in PLP injected animals

| | | | | | |
|---|---|---|---|---|---|
| | Incidence | | | | |
| Treatment | (#dead) | MMS | MDD | MDO | Mean score |

Assay 1^{(a)}

| | | | | | |
|---|---|---|---|---|---|
| Control (saline IV) | 10/10 (3) | 3.9 ± 0.526 | 9.8 ± 1.2 | 11 ± 0 | 2.3 ± 0.223 |
| Copaxone | 8/10 (3) | 2.9 ± 0.69 | 8.1 ± 1.72 | 9.9 ± 1.74 | 1.8 ± 0.219 |
| Betaferon | 8/10 (3) | 3.15 ± 0.753 | 7.7 ± 1.51 | 10.3 ± 1.84 | 1.8 ± 0.245 |
| SSL-TMN (i.v) | 8/10(1) | 2.35 ± 0.6 | 5.3 ± 1.54 | 11.9 ± 2.29 | 1.1 ± 0.178 |

Assay 2^{(a)}

| | | | | | |
|---|---|---|---|---|---|
| Control (saline sc) | 8/9 (1) | 3.89 ± 0.539 | 11.1 ± 1.51 | 11.8 ± 1.61 | 1.76 ± 0.149 |
| SSL-TMN (s.c) | 4/5(1) | 3.67 ± 0.88 | 4.67 ± 1.2 | 14 ± 1.5 | 0.8 ± 0.2 |

^{(a)}two identical assays conducted at different times
MMS = mean maximal score; MDD = mean disease duration (days); MDO = mean day of onset; SSL-TMN = TMN loaded in sterically stabilized liposome composed of EPC:Chol:²⁰⁰⁰PEG-DSPE

TABLE 4B
clinical signs scores in PLP injected animals

| | Incidence | | | | |
|---|---|---|---|---|---|
| Treatment | (#dead) | MMS | MDD | MDO | Mean score |
| Control (saline | 8/8 (4) | 4.6 ± 0.42 | 21 ± 0.8 | 15 ± 0.3 | 3.9 ± 0.16 |
| s.c.) | | | | | |
| EPC-SSL-TMN | 6/7(0) | 2.7 ± 0.56 | 17 ± 0.3 | 13.3 ± 2.3 | 1.37 ± 0.11 |
| EPC-SSL^{(a)} | 5/5 (0) | 4.5 ± 0.3 | 21 ± 0.6 | 14 ± 0.4 | 4.2 ± 0.28 |
| TMN Free | 5/5 (1) | 3.9 ± 0.53 | 19 ± 1.2 | 14 ± 0.5 | 3.5 ± 0.21 |

^{(a)}SSL liposome with no encapsulated TMN
^{(b)}Same concentration as encapsulated in the liposomes

TABLE 4C
clinical signs scores in PLP injected animals

| | Incidence | | | | |
|---|---|---|---|---|---|
| Treatment | (#dead) | MMS | MDD | MDO | Mean score |
| Control (saline | 3/5(0) | 5.5 ± 0.29 | 7.25 ± 0.48 | 12.2 ± 0.7 | 2.6 ± 0.37 |
| s.c) | | | | | |
| EPC-SSL-TMN | 4/5(1) | 3.67 ± 0.88 | 4.67 ± 1.2 | 14 ± 1.5 | 0.8 ± 0.2 |
| HSPC-SSL- | 4/5 (1) | 4.1 ± 0.8 | 6.25 ± 1.37 | 11.5 ± 0.5 | 1.75 ± 0.3 |
| TMN | | | | | |
| EPC-SSL^{(a)} | 5/5 (0) | 5.3 | 9 | 11 | 3.3 |
| HSPC-SSL^{(a)} | 5/5 (1) | 5.1 | 8.7 | 11 | 3.5 |

^{(a)}SSL liposome with no encapsulated TMN

The results above and in FIG. 2 demonstrate that intravenous administration of sterically stabilized TMN (SSL-TMN, 80 nm in diameter) was more effective in reducing the clinical signs of MS as compared to the signs observed with conventional medications (Copaxone and Betaferon) or as compared to empty SSL liposomes (EPC or HSPC) or free TMN, the empty liposomes or free TMN having no observed effect against the disease.

B. Biodistribution Studies

Mice received 0.1 ml [³H] Cholesteryl hexadecyl ether (0.5 μCi/μmol phospholipid) labelled TMN-SSL i.v injection At different time points post injection (1, 6, 16, 24, 48 and 72 hours after the [³H] Cholesteryl hexadecyl ether liposomal injection) the mice were anesthetized with ether inlialation, bled from the orbital sinus, and immediately sacrificed for removal of brain, heat, lungs, liver, spleen, stomach and kidney. Plasma was separated by centrifugation.

Organ samples were homogenized in a Polytron homogenizer (Kinematica, Lutzem, Switzerland) in 2% Triton X-100 (1:2, organ:Triton X-100 solution), cooled and heated several times to destroy the lipid membrane. The plasma samples were mixed 1:1 with 2% Triton X-100 to give the 1% Triton X-100 in the tested sample and also cooled and heated several times. It was determined that under such conditions intact liposomes released all their TMN content.

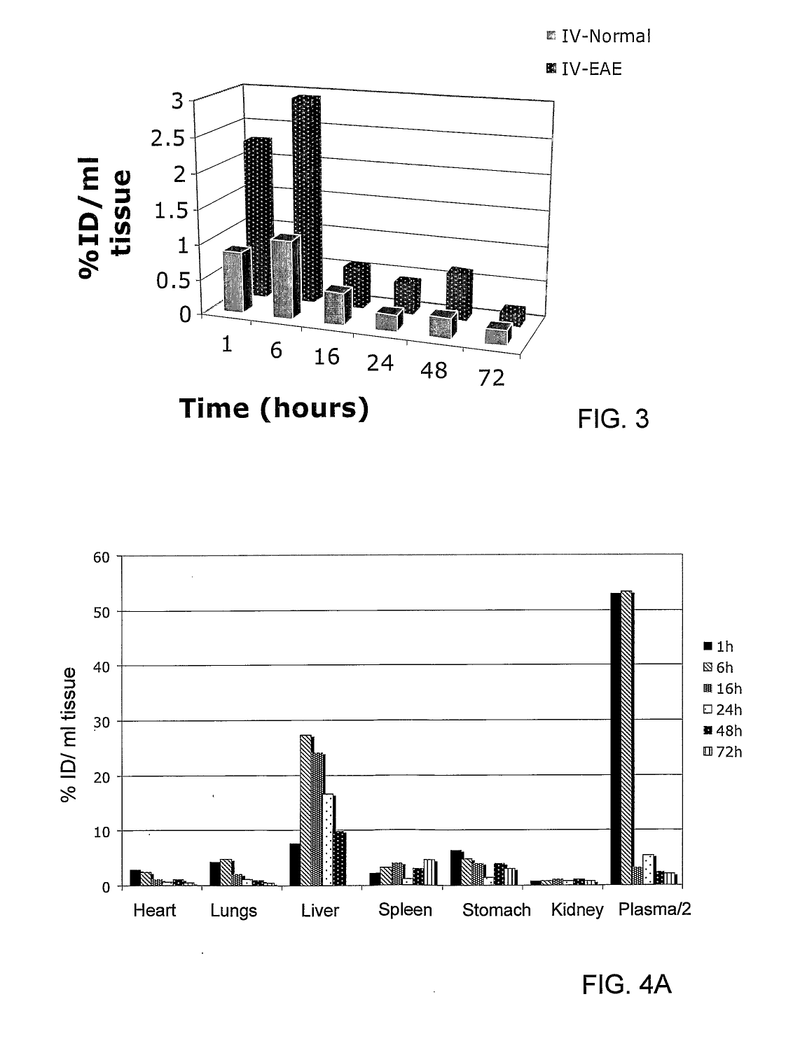

Sample duplicates of 100 μl were burned in a Sample Oxidizer (Model 307, Packard Instrument Co., Meridien, Conn.) left overnight in a dark, cool place and measured by β-counting (KONTRON Liquid Scintillation Counter), reflecting the amount of liposomal TMN in each organ. FIG. 3 presents the percent of absorbance per ml tissue in healthy and EAE induced mice, after treatment with liposomal TMN (EPC:Chol²⁰⁰⁰PEG-DSPE). Specifically shown is that [³H] Cholesteryl hexadecyl ether SSL-TMN liposomes penetration was higher in brains of diseased (EAE) mice than in that of healthy mice, particularly during the first 6 hours after injection of [³H] Cholesteryl hexadecyl ether SSL-TMN liposomes. It is assumed that this is a result of a disruption in the blood brain barrier (BBB) which is common with MS and similar neurodegenerative disorders.

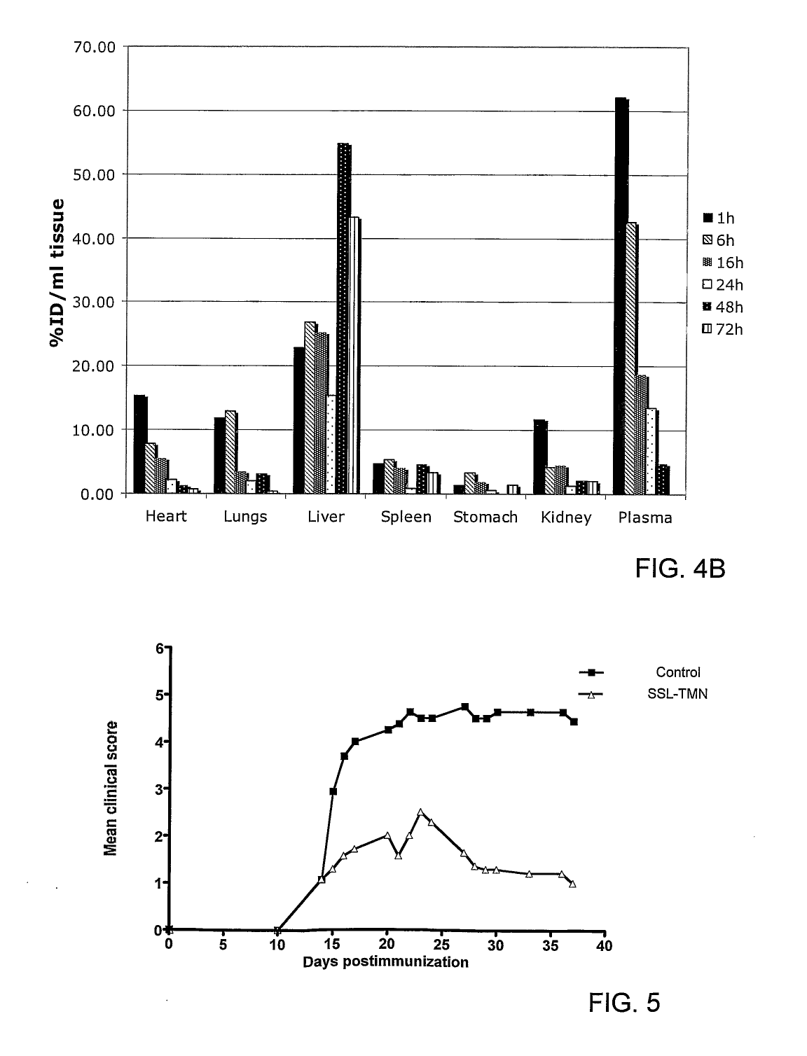

The difference in tissue distribution of the liposomal TMN in healthy and diseases animal models is shown in FIG. 4A-4B respectively.

C. Induction of Chronic EAE Using MOG (Myelin Oligodendrocyte Glycoprotein)

Induction of chronic EAE using MOG 35-55 peptide was performed as described in [Offen D et al J Mol Neurosci. 15(3):167-76 (2000)]. In general, female C57B1/6 mice were inoculated (s.c. injection in the right flank) with an encephalitogenic emulsion (MOG plus CFA enriched with MT (mycobacterium tuberculosis). Pertussis toxin was injected i.p (250 ng/mouse) on the day of inoculation and 48 hrs later. A boost of the MOG emulsion was injected s.c. in the right flank one week after first injection. On day 10, each mouse was injected (i.v.) with SSL-TMN formulation or with the control solution. The animals were kept in SPF conditions and given food and water ad libitun. Treatment was terminated on day 30.

For treatment, the animals (10 mice per group) were divided into groups and treated as summarized in Table 5 below.

TABLE 5
Schedule of treatment

| Group | Treatment formulation | Dose | Regime of administration |
|---|---|---|---|
| 1 | Saline | 45 mg/kg | i.v. injection × 3/week |
| 2 | SSL-TMN | 8.5 mg/kg | i.v. injection × 3/week |

The mice were observed daily from the 10th day post-EAE induction (first injection of MOG) and the EAE clinical signs were scored according to the Table 3 shown above. The results are presented in Table 6 and FIG. 5.

TABLE 6
clinical signs scores in MOG injected animals

| | Incidence | | | | |
|---|---|---|---|---|---|
| Compound | (#dead) | MMS | MDD | MDO | Mean score |
| Control (Saline) | 8/8(4) | 4.6 ± 0.42 | 21 ± 0.8 | 15 ± 0.3 | 3.9 ± 0.16 |
| EPC:Chol:²⁰⁰⁰PEG-DSPE | 6/7 (0) | 2.7 ± 0.56 | 17 ± 0.3 | 13.3 ± 2.33 | 1.37 ± 0.11 |

Table 6 and FIG. 5 show that SSL-TMN was effective in reducing the clinical signs of MS also in MOG induced animal model of the chronic disease as compared to the control (saline)

### EXAMPLE 2B

### Parkinson Disease

For determining the effect of the liposomal TMN formulation in treating Parkinson disease the conventional 6-Hydroxydopamine (6-OHDA) Parkinson animal model was used [Hastings T G et al; Proc. Natl. Acad. Sci USA 93:195619-195661 (1996)]. This model is characterized by the unilateral injection of 6-OHDA into the substantia nigra with the ulterior accumulation of the toxin (6-OHDA) into dopaminergic neurons leading to their death presumably mediated by oxidative stress. In brief, 6-OHDA (8 μg/rat) was stereotaxically injected in 4 μl into the right substantia nigra of male Sprague-Dawley rats (weighing 250-280 g; coordinates of injection: P=4.8, L=1.7, H=−8.6 from bregma). Eighteen days after 6-hydroxydopamine injection, rats were selected for transplantation if they had >350 rotations per hour after s.c. injection of apomorphine (25 mg/100 g body weight) and, if 2 days later, they also had >360 (mean 520±38) rotations per hour after i.p. injection of D-amphetamine (4 mg/kg).

The effectiveness of the lesion in the substantia nigra was evaluated with the stepping test [Olsson M et al; J. neurosci 15(5):3863-3875 (1995)]. This test determines motor initiation deficits in the forelimbs of the rats, analogous to limb akinesia and gait problem in Parkinson patients The 6-OHDA lesion profoundly affect the adjusting steps, it means that there is a significant impairment in the left paw performance (contralateral to the lesion) which results in a dragging paw when the rat is moved sideways by the experimenter. By contrast right paw is unaffected. Animals receiving SSL-TMN (EPC:Chol:²⁰⁰⁰PEG-DSPE) show a significant increase in the adjusting steps number in contrast with the control 6OHDA animals The number of stepping adjustments was counted for each forelimb during slow sideway movements in forehand directions over a standard flat surface. The stepping adjustments test was performed twice for each forelimb after SSL-TMN injection and the SSL-TMN treated animals restored the number of adjusting steps to a level similar from that seen in intact control animals (animals that didn't receive 6-OHDA). The stepping test was repeated at least three times between days 15 and 20 after the lesion in all the rats. Only those rats treated with 6-OHDA and which showed less than two adjusting steps with the forelimb contralateral to the lesion during each trial were selected for treatment.

Specifically, rats were divided into two groups:

Group I—rats receiving treatment with 1 ml SSL-TMN (either i.v. or s.c. injection) 2 and 4 days before induction of the disease with 6-OHDA

Group II—rats receiving treatment with 1 ml SSL-TMN 2, 4 and 7 days after the induction of the disease with 6-OHDA.

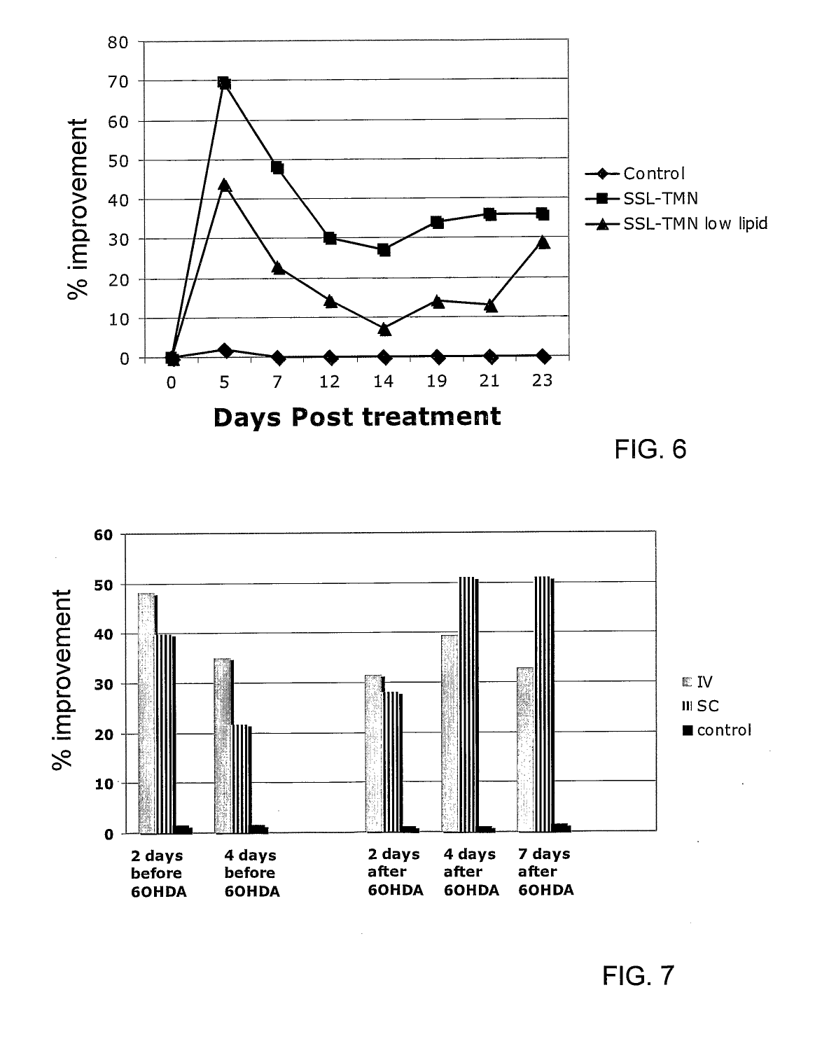

The rats were observed daily from the day of induction (day 0), and the clinical signs were scored. Results are presented in FIG. 6.

The behavior of the rats was also examined through the stepping test described above. Specifically, the percent of improvement in the stepping adjustment test (left paw over the right paw X100) was scored, the results of which are shown in FIG. 7.

1-51. (canceled)

52. A method of treating a subject having, or in disposition of developing, a neurodegenerative condition, the method comprising administering to said subject a pharmaceutical formulation comprising an amount of an amphipathic weak base, the amount being effective to treat or prevent the development of a neurodegenerative condition, wherein said amphipathic weak base has one or more of the following characteristics: (i) it has pKa below 11.0; (ii) in an n-octanol/buffer (aqueous phase) system having a pH of 7.0, it has a partition coefficient in the range between 0.001 and 5.0; (iii) it exhibits an antioxidative activity; (iv) it exhibits a pro-apoptotic activity.

53. The method of claim 52, wherein said amphipathic weak base is characterized by a pKa below 11.0 and a partition coefficient in the range between 0.001 and 5.0.

54. The pharmaceutical formulation of claim 52, wherein said partition coefficient is in the range of between 0.005 and 0.5.

55. The method of claim 52, wherein said formulation comprises a liposome encapsulating said amphipathic weak base.

56. The method of claim 55, wherein said liposome comprises a combination of phospholipid, cholesterol and a lipopolymer.

57. The method of claim 56, wherein said phospholipid is egg phophatidylcholine (EPC), 1-palmitoyl-2-oleoylphosphatidyl choline (POPC), distearoylphosphatidylcholine (DSPC) or hydrogenated soy phosphatidylcholine (HSPC).

58. The method of claim 56, wherein said combination comprises EPC:Chol:²⁰⁰⁰PEG-DSPE or HSPC:Chol:²⁰⁰⁰PEG-DSPE at a mole ratio of 54:41:5.

59. The method of claim 52, wherein said amphipathic weak base is a cyclic nitroxide.

60. The method of claim 52, wherein said amphipathic weak base is TMN.

61. The method of claim 52, for the treatment of a neurodegenerative condition associated with abnormal deterioration of the nervous system resulting in the dysfunction of the system.

62. The method of claim 52, wherein said neurodegenerative condition is selected from demyelinating and neuroautoimmune diseases.

63. The method of claim 52, wherein said neurodegenerative condition is acute, chronic, progressive, and relapsing remitting multiple sclerosis.

64. The method of claim 52, wherein said neurodegenerative condition is selected from neurodegenerative disorders.

65. The method of claim 64, wherein said neurodegenerative disorder is Parkinson's disease.

66. The method of claim 52, comprising parenteral administration of said pharmaceutical formulation.

67. The method of claim 66, wherein said parenteral administration comprises administration by injection.

68. The method of claim 56, for the treatment of a neurodegenerative condition associated with abnormal deterioration of the nervous system resulting in the dysfunction of the system.

69. The method of claim 56, wherein said neurodegenerative condition is selected from demyelinating and neuroautoimmune diseases.

70. The method of claim 56, wherein said neurodegenerative condition is acute, chronic, progressive, and relapsing remitting multiple sclerosis.

71. The method of claim 56, wherein said neurodegenerative condition is selected from neurodegenerative disorders.

72. The method of claim 71, wherein said neurodegenerative disorder is Parkinson's disease.
